(19) United States
(12) Patent Application Publication
(10) Pub. No.: US 2021/0052749 A1
(43) Pub. Date: Feb. 25, 2021

(54) AMPHIPHILIC POLYMER NANO MICELLE CONTAINING POLY-3,4-DIHYDROXYPHENYLALANINE CHELATED FERRIC IONS

(71) Applicant: ZHEJIANG UNIVERSITY, Hangzhou (CN)

(72) Inventors: Jihong SUN, HANGZHOU (CN); Jun LING, HANGZHOU (CN); Jiayu CEN, HANGZHOU (CN); Yuedong MIAO, HANGZHOU (CN)

(21) Appl. No.: 17/088,566

(22) Filed: Nov. 3, 2020

Related U.S. Application Data
(63) Continuation of application No. PCT/CN2019/086586, filed on May 13, 2019.

(30) Foreign Application Priority Data
May 16, 2018 (CN) 201810468018.2

(51) Int. Cl. A61K 49/18; A61B 5/055; A61K 49/12; A61K 49/10

(52) U.S. Cl. CPC A61K 49/1809; A61B 5/055; B82Y 30/00; A61K 49/101; A61K 49/1857; A61K 49/124

(57) ABSTRACT

The disclosure discloses an amphiphilic polymer nano micelle containing poly-3,4-dihydroxyphenylalanine chelated ferric ions, in which ferric ions are chelated with a catechol structure on a side chain of a biodegradable poly-3,4-dihydroxyphenylalanine block. The disclosure also provides a method for preparing the above micelle, comprising: complexing an amphiphilic polymer containing poly-3,4-dihydroxyphenylalanine with a ferric ion compound, and obtaining the amphiphilic polymer nano micelle containing poly-3,4-dihydroxyphenylalanine chelated ferric ions through a solvent replacement method. The micelle prepared by the disclosure is used as a Fe³⁺ magnetic resonance Ti imaging contrast agent, which can avoid toxic or side effects caused by a traditional gadolinium reagent, has a longitudinal relaxation rate of 5.6 mM⁻¹·s⁻¹, can cycle for 150 min in a mice body, and has an obvious imaging effect and a far higher comprehensive performance than that of a commercial gadolinium contrast agent, and as well as a promising application prospect.

### CROSS-REFERENCE TO RELATED APPLICATIONS

This application is a continuation of International Patent Application No. PCT/CN2019/086586 with a filing date of May 13, 2019, designating the United States, now pending, and further claims priority to Chinese Patent Application No. 201810468018.2 with a filing date of May 16, 2018. The content of the aforementioned applications, including any intervening amendments thereto, are incorporated herein by reference.

### TECHNICAL FIELD

The disclosure belongs to the field of magnetic resonance imaging, and particularly relates to an amphiphilic polymer nano micelle containing poly-3,4-dihydroxyphenylalanine chelated ferric ions, and use.

### BACKGROUND OF THE DISCLOSURE

Magnetic resonance imaging (MRI) is to detect an electromagnet wave emitted from a body by utilizing a nuclear magnetic resonance principle and a high-frequency gradient magnetic field so as to draw a structure image inside an object. Since MRI is a noninvasive, real-time, three-dimensional-structural imaging method, and capable of providing high-resolution Information of soft tissue, it is one of the most advanced clinical medical examination technologies (Nat. Rev. Neurosci. 2007, 8, 700-711).

To improve contrast degrees between normal tissues and lesions, a magnetic resonance contrast agent is needed in over 30% diagnosis. The magnetic resonance contrast agent currently applied clinically is mainly a complex of gadolinium ion: Gd-DTPA (trade name: Magnevist), Gd-DATA (trade name: Dotarem) and the like (J. Magn. Recon. Imaging 2009, 30, 1259-1267), however, the half-time elimination of these small molecule gadolinium contrast agents in blood and tissues is generally within 30 min, with relatively short residence time, which do influence use of them in clinic (Anal. Chim. Acta. 2013, 764, 1-16).

In addition, many clinical researches indicate that a Gd³⁺ reagent has biotoxicity: it enriches in human skins and viscera, and increases a risk that the renal function defect in patients is further deteriorated into a rare and lethal disease: nephrogenic systemic fibrosis (NSF) (Nephrol. Dial. Transpl. 2006, 21, 1745-1745). Besides, the Gd³⁺ reagent can be deposited in brain tissues for a long term (Radiology. 2017, 285, 546-554).

Therefore, it is urgent to develop a low-toxic or non-toxic magnetic resonance contrast agent.

At present, the low-toxic or non-toxic non-gadolinium magnetic resonance contrast agent used clinically is mainly a T₂ imaging contrast agent represented by Feridex (superparamagnetic ferric oxide injection). Compared with the gadolinium contrast agent, the T₂ imaging contrast agent has the following advantages: 1) it is easily swallowed by a reticuloendothelial system in the body, used for detection and diagnosis of lesions of liver, spleen and other targeted parts; 2) metal Fe per unit on a T₂WI sequence can produce more signal intensity changes, high relaxation performance and more sensitive detection; 3) it has biodegradability in the body and enters into in-vivo Fe cycle via a normal metabolism pathway in cells. The T₂ imaging contrast agent has the following disadvantages that, one the one hand, the T2 imaging contrast agent is difficultly distinguished from low-signal substances such as a gas, bone cortex and in-vivo Fe deposition substance; on the other hand, the application scope of the contrast agent is, limited and restricted by the reticuloendothelial system. To change the biocompatibility and prolong the circulation time in blood, it is necessary to modify the surfaces of ferric oxide nano particles.

For example, patent CN102552944B discloses a nasopharyngeal carcinoma targeted magnetic resonance contrast agent. Superparamagnetic Fe₃O₄ with a particle size of about 10-15 nm is synthesized by a chemical co-precipitation method. APTES is used to coat or connect to the surface of superparamagnetic Fe₃O₄ having the particle size of 10-15 nm so that the surface is aminated to obtain Fe₃O₄-APTES surface modified particles. Polyethylene glycol is used as a link arm between Fe₃O₄-APTES and EB virus latent membrane protein 1 monoclonal antibody (LMP1, Clone CS.1-4) to obtain stably dispersed Fe₃O₄-APTES-PEG-MP1, Clone CS.1-4 colloidial solution as a magnetic resonance contrast agent.

However, the surface modification of ferric oxide nano particles is usually tedious in steps, time-consuming and high in cost, and T₂ imaging magnetic resonance contrast agent has been basically eliminated. Therefore, it is necessary to develop a new magnetic resonance T₁ imaging contrast agent which can replace gadolinium.

### SUMMARY OF DISCLOSURE

The disclosure provides an amphiphilic polymer nano micelle containing poly-3,4-dihydroxyphenylalanine chelated ferric ions, which has excellent biocompatibility and biodegradability. The amphiphilic polymer nano micelle is used as or prepared into the Fe³⁺ magnetic resonance contrast agent, which is a new non-toxic efficient non-gadolinium T₁ magnetic resonance imaging contrast agent that has high relaxation performance and long in-vivo circulation time.

Provided is an amphiphilic polymer nano micelle containing poly-3,4-dihydroxyphenylalanine chelated ferric ions, wherein the hydrophobic block in the amphiphilic polymer forming the nano micelle is a poly-3,4-dihydroxyphenylalanine block having biodegradability; the amphiphilic polymer nano micelle containing poly-3,4-dihydroxyphenylalanine uses the catechol functional group on the side chain of the biodegradable poly-3,4-dihydroxyphenylalanine block to chelate ferric ions, and a chelating bond is as shown in the following formula:

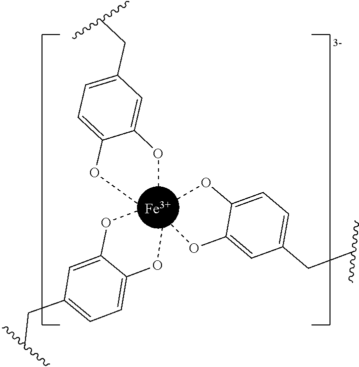

The disclosure aims to provide an amphiphilic polymer nano micelle having good biocompatibility and containing poly-3,4-dihydroxyphenylalanine chelated ferric ions. Since the hydrophobic block is a poly-3,4-dihydroxyphenylalanine block, the amphiphilic polymer nano micelle is non-toxic and good in biocompatibility. Therefore, in a broad sense, it is feasible for the hydrophilic block in the amphiphilic polymer to select the polymer block having good biocompatibility. Preferably, the hydrophilic block is a polysarcosine block, a polyethylene glycol block, a polyoligoethyleneglycol methacrylate, a polyvinyl alcohol block or a polyacrylic acid block. Further preferably, the hydrophilic block is a polysarcosine block, a polyethylene glycol block, or polyoligoethyleneglycol methacrylate block.

The degree of polymerization of the polymer affects the solubility and flexibility of the polymer, and the degree of polymerization of the hydrophobic block and the hydrophilic block of the amphiphilic polymer affect the stability of the formed micelles. Through comprehensive consideration, the chain length of the poly-3,4-dihydroxyphenylatanine block is preferably 1˜500; the chain length of the hydrophilic block is preferably 1˜1500.

The micelle of the disclosure are a micelle in a broad sense: because the hydrophobic part of the amphiphilic polymer has a small affinity with water, but the attraction between the hydrophilic parts is large, when the concentration of the amphiphilic polymer in water reaches a certain concentration, the hydrophobic parts of the amphiphilic polymer attract with each other to be associated together to form an association having various shapes (such as a sphere, a layered shape and a rod shape). This association is the micelle in a broad sense. Correspondingly, the amphiphilic polymer comprises amphiphilic polymers having various topological structures, such as diblock, triblock, multiblock, random, star shaped, ring-shaped and grafted polymers containing poly-3,4-dihydroxyphenylalanine blocks.

The representative examples of the amphiphilic polymer nano r beetle containing poly-3,4-dihydroxyphenylalanine include the following structures:

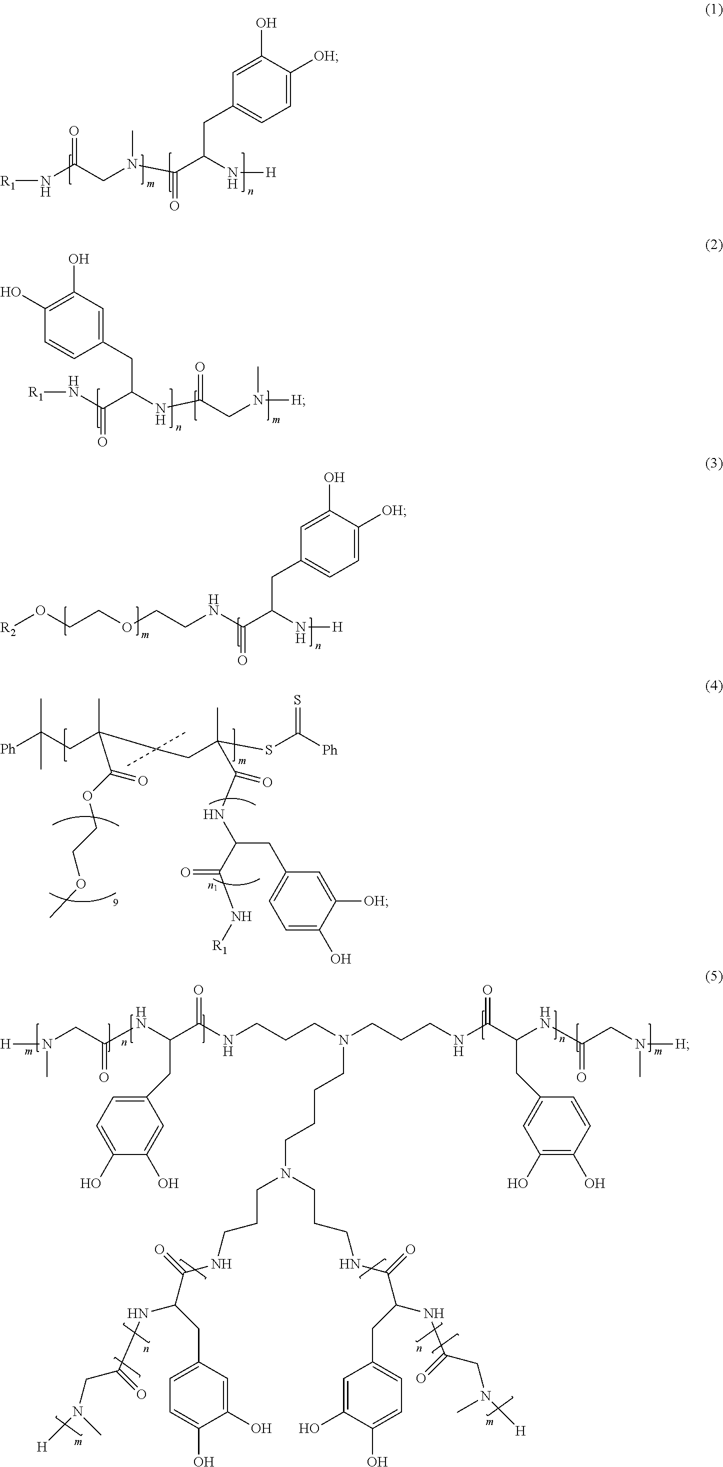

wherein, R₁ is independently selected from alkyl, benzyl and a silyl group; R₂ is independently selected from alkyl; m is an integer of 1˜1500, n is an integer of 1˜500, and n₁ is an integer of 1˜200.

The smaller the degree of polymerization is, the easier the synthesis of amphiphilic polymer is, the shorter the polymerization time is, and the stability of the formed micelles is relatively good, Therefore, further preferably, the chain length of poly-3,4-dihydroxyphenylalanine is between 5 and 50, and the chain length of polysarcosine or polyethylene glycol is between 5 and 200.

The disclosure also provides a method for preparing the amphiphilic polymer nano micelles containing poly-3,4-dihydroxyphenylatanine chelated ferric ions, which is simple and feasible, and comprises:

(1) Amphiphilic polymers containing poly-3,4-dihydroxyphenylalanines are synthesized by referring to preparation methods of poly(amino acid) reported in a document (Miaoer Yru, and Timothy J. Deming, synthetic polypeptide mimics of marine adhesives, macrolecules, 1998, 31 (15), 4739-4745) in combination with the existing copolymerization method;

(2) The amphiphilic polymer containing poly-3,4-dihydroxyphenylalanine obtained in step 1 was complexed with a ferric ion compound, and the amphiphilic polymer nano micelle containing poly-3,4-dihydroxyphenylalanine chelating ferric ion was obtained by a solvent displacement method.

The ferric ion compound is one or more of ferric nitrate, ferric sulfate, ferric chloride, ferric bromide and the like. Further preferably, the ferric ion compound is ferric nitrate.

The disclosure also provides use of the above amphiphilic polymer nano micelle containing poly-3,4-dihydroxyphenylalanine chelated ferric ions serving as or preparing a magnetic resonance T₁ imaging contrast agent in the field of magnetic resonance imaging.

When the amphiphilic polymer nano micelle containing poly-3,4-dihydroxyphenylalanine chelated ferric ions is used to prepare the magnetic resonance contrast agent, pharmaceutical excipients, diluents or ingredients need to be added. The magnetic resonance T₁ imaging contrast agent provided by the disclosure can be administered orally or parenterally (mainly intravenous administration) to human and non-human mammals (such as mice, rats, hamsters, rabbits, cats, dogs and pigs). The dose varies with administration objects, MRI sites, dosage forms and administration routes.

As long as the particle size of the amphiphilic polymer nano micelle containing poly-3,4-dihydroxyphenylalanine chelated ferric ions serving as or preparing the magnetic resonance contrast agent reaches a nano scale, its good dispersion performance can sufficiently avoid the problem that the magnetic resonance contrast agent particles easily result in death of animals due to agglomeration. Further preferably, the average particle size is 20˜200 nm.

When the amphiphilic polymer nano micelle containing poly-3,4-dihydroxyphenylalanine chelated ferric ions serves as or prepares the magnetic resonance contrast agent, magnetic resonance imaging can be realized as long as there are chelated ferric ions. From the perspective of optimizing the imaging effect, the amount of ferric ions is preferably 10˜1000 ppm.

The amphiphilic polymer nano micelle containing poly-3,4-dihydroxyphenylalanine chelated ferric ions serves as prepare the magnetic resonance contrast agent. The experimental results show that it has a longitudinal relaxation rate (r₁) of 5.6 mM⁻¹·s⁻¹, circulation time in the mice body being up to 150 min, an obvious imaging effect and comprehensive performance being far higher than that of the commercial gadolinium contrast agent. It is expected to replace the traditional Gd³⁺ contrast agent in diagnostic imaging and becomes a new non-toxic and efficient gadolinium-free T₁ imaging MRI contrast agent.

The amphiphilic polymer nano micelle containing poly-3,4-dihydroxyphenylalanine chelated ferric ions can serve as the magnetic resonance T₁ imaging contrast agent, which can not only target at the liver and spleen and other reticuloendothelial systems to realize T₁ enhanced imaging, but also realize angiography, that is, vascular imaging can be realized; moreover, the magnetic resonance T₁ imaging contrast agent provided by the disclosure can be distributed throughout the body via blood circulation and enter into the tissue organs in the whole body to realize T₁ enhanced imaging, breaks through the limitation that the traditional Fe magnetic resonance contrast agents can only target at the reticuloendothelial system such as liver and spleen, and broadens the medical application range of the Fe magnetic resonance contrast agents, and has a good application prospect.

Compared with the prior art, the disclosure has the advantages:

(1) The amphiphilic polymer nano micelle containing poly-3,4-dihydroxyphenylalanine chelated ferric ions provided by the disclosure can be used as a Fe³⁺ magnetic resonance contrast agent, and has extremely low biotoxicity; moreover, the micelle has good dispersion performance to avoid the problem that the magnetic resonance contrast agent particles easily result in death of animals due to agglomeration.

(2) The amphiphilic polymer containing poly-3,4-dihydroxyphenylalanine used in the disclosure is a polyamino acid material, which has excellent biocompatibility and biodegradability, and has obvious potential application advantages.

(3) The amphiphilic polymer nano micelle containing poly-3,4-dihydroxyphenylalanine chelated ferric ions provided by the disclosure serves as the magnetic resonance T₁ imaging contrast agent. It has a longitudinal relaxation rate (r₁) of 5.6 mM⁻¹·s⁻¹, circulation time in the mice body being up to 150 min, an obvious imaging effect and comprehensive performance being far higher than that of the commercial gadolinium contrast agent.

(4) The amphiphilic polymer nano micelle containing poly-3,4-dihydroxyphenylalanine chelated ferric ions provided by the disclosure is simple and feasible in preparation method, and suitable for industrial production.

### DESCRIPTION OF THE DRAWINGS

FIG. 1 shows proton nuclear magnetic resonance (NMR) spectra of poly-3,4-dihydroxyphenylalanine-polysarcosine block copolymer (A) and polysarcosine (B) prepared in example 1 of the disclosure.

FIG. 2 is a TEM diagram of an amphiphilic polymer nano micelle containing poly-3,4-dihydroxyphenylalanine chelated ferric ions prepared in example 1 of the disclosure.

FIG. 3 is a dynamic light scattering diagram of an amphiphilic polymer nano micelle containing poly-3,4-dihydroxyphenylalanine chelated ferric ions prepared in example 1 of the disclosure.

FIG. 4 shows in-vitro experimental results of a relationship between longitudinal relaxation time (T₁) and ferric ion concentration of an amphiphilic polymer nano micelle containing poly-3,4-dihydroxyphenylalanine chelated ferric ions prepared in example 1 of the disclosure.

FIG. 5 is magnetic resonance angiography of an amphiphilic polymer nano micelle containing poly-3,4-dihydroxyphenylalanine chelated ferric ions as a Fe′ magnetic resonance contrast agent in a mice body prepared in example 1 of the disclosure.

FIG. 6 is a cytotoxicity test diagram of an amphiphilic polymer nano micelle containing poly-3,4-dihydroxyphenylalanine chelated ferric ions prepared in example 1 of the disclosure.

FIG. 7 is a proton NMR spectrum of a poly-3,4-dihydroxyphenylalanine-polyoligoethyleneglycol methacrylate grafted polymer prepared in example 3 of the disclosure.

FIG. 8 is a dynamic light scattering diagram of an amphiphilic polymer nano micelle containing poly-3,4-dihydroxyphenylalanine chelated ferric ions prepared in example 3 of the disclosure.

### DETAILED DESCRIPTION OF PREFERRED EMBODIMENTS

In order to further understand the disclosure, the preparation and use of an amphiphilic polymer nano micelle containing poly-3,4-dihydroxyphenylalanine chelated ferric ions provided by the disclosure are described in detail in combination with embodiments. However, the disclosure is not limited to these embodiments. The non-essential improvements and adjustments made by those skilled in the art under the core guiding ideology of the disclosure still belong to the protective cope of the disclosure.

The characterization method involved in embodiments of the disclosure is briefly introduced as follows:

The NMR spectra were determined at 25° C. on Bruker Avarice DMX 00 superconducting NMR instrument. Deuterated DMSO was a solvent and tetramethylsilane (TMS) was an internal standard.

The hydrodynamic diameter of the polymer nano micelle in solution was detected by Zetasizer Nano series (Malvern instruments) detector. The wavelength was measured as 657 nm and a fixed angle was 90°. Each sample was tested three times in parallel.

The particle size and morphology of the nano micelle were observed by HITACHI HT7700 transmission electron microscope, and an acceleration voltage was 100 KV.

The relaxation rate r₁ of T₁ weighted magnetic resonance imaging of metal chelated polymer nano particles was measured on a 3.0 T magnetic resonance imager (Aigna HDxt, GE Medical Systems, Milwaukee, Wis., USA).

The cytotoxicity test of metal chelating polymer nano particles was realized through MTT method. The test cells were mice embryonic fibroblasts (NIH 3T3), and the test results were obtained by ELIA SA (Thermo Fisher. Scientific (Waltham, Mass.).

### Example 1

(1) The structural formula of poly-3,4-dihydroxyphenylalanine-poly(asarcosine) block copolymer (PDOPA-b-PSar) is as follows:

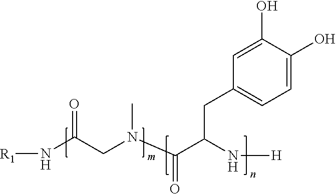

wherein, R₁ is benzyl, M=5˜200, n=5˜50;

The specific synthesis steps include:

Sarcosine NCA was added into Schlenk bottle, dissolved with DMF, and then DMF solution of benzylamine was added. The molar ratio of sarcosine NCA to benzylamine was (5˜200):1, and the above materials reacted for 1 day at room temperature. Then DMF solution of DOPA NCA protected by benzyloxycarbonyl (CBZ) was added. The molar ratio of DOPA NCA to benzylamine was (5˜50):1, and the above materials reacted for 1 day at room temperature. The polymer solution was poured into ether to be precipitated and filtered. The obtained polymer was dried in vacuum for 1 day to obtain the CBZ-protected poly-3,4-dihydroxyphenylalanine-polysarcosine block copolymer was obtained.

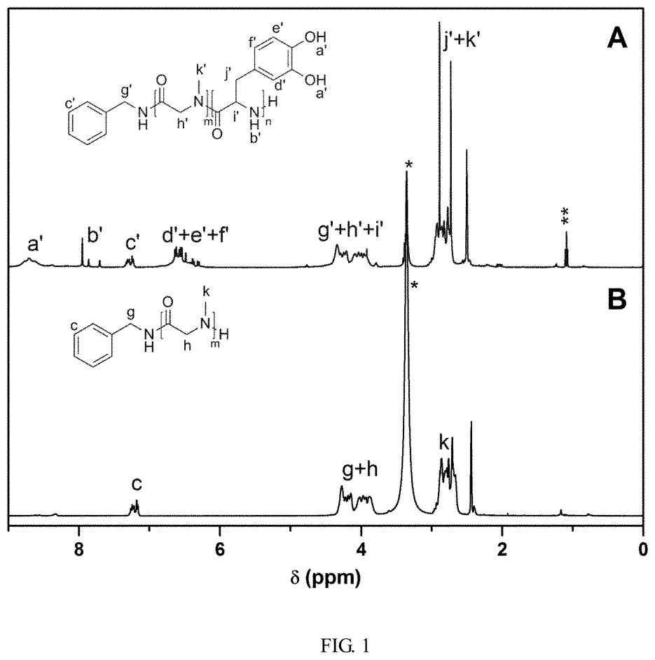

300 mg of block copolymer was dissolved into 3 mL of trifluoroacetic acid, and 4-fold equivalent weight of hydrogen bromide acetic acid solution (33%) was added. After reaction for 3 hours, the reactant was precipitated with ether and filtered. The obtained polymer was dried in vacuum for 1 day to obtain poly-3,4-dihydroxyphenylalanine-polysarcosine block copolymer. The yield was 89%. The NMR spectrum of the polymer is shown in FIG. 1.

(2) 7.7 mg of weighed PDOPA-b-PSar was dissolved with DMF to be prepared into a solution, and then the DMF solution containing 1.69 mg of Fe(NO₃)₃.9H₂O was added slowly. After mixing evenly, the obtained mixture solution was dialyzed in deionized water for 48 hours to obtain the micelle solution. The micelle solution was subjected to metered volume and used after being filtered with a filter film having a pore size of 0.45 μM. After conducting metered volume, the Fe³⁺ concentration was 94 mg/L.

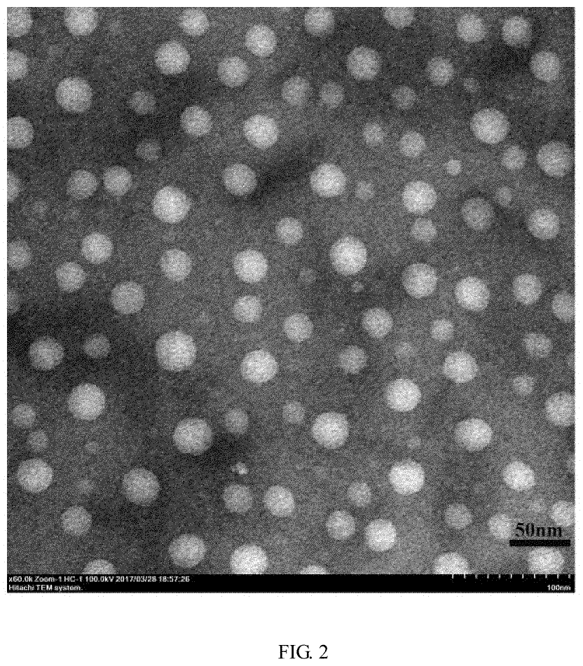

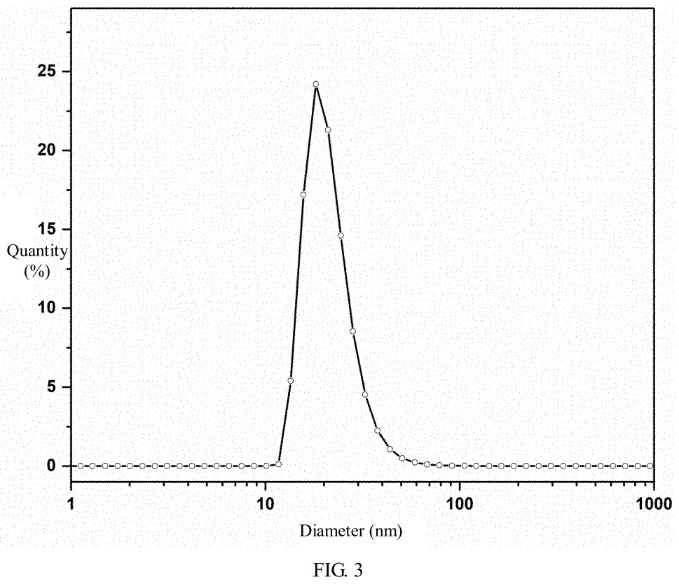

The TEM of the micelles is shown in FIG. 2, and DLS test results are shown in FIG. 3. It can be seen from FIGS. 2 and 3 that the average particle size of the prepared micelle is 20 nm.

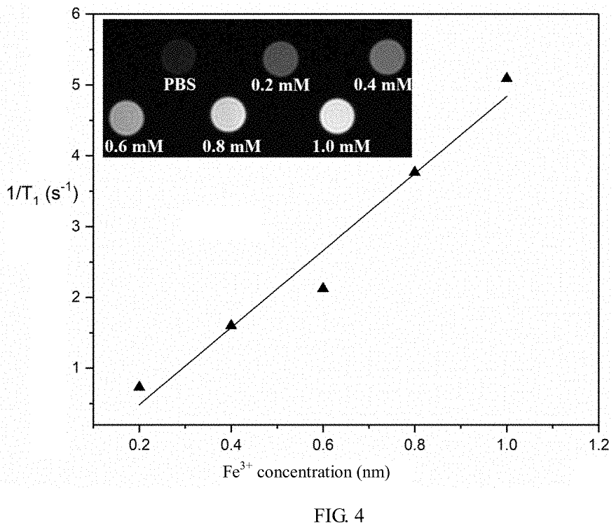

The in-vitro experimental results of the relationship between the longitudinal relaxation time (T1) of the micelle and the ferric ion concentration are shown in FIG. 4. It can be seen from FIG. 4 that the longitudinal relaxation rate of the micelle is 5.6 mM⁻⁴·s⁻¹, which is higher than that of the commercial gadolinium contrast agent (such as Gd-DTPA), showing excellent in-vitro magnetic resonance enhancement ability.

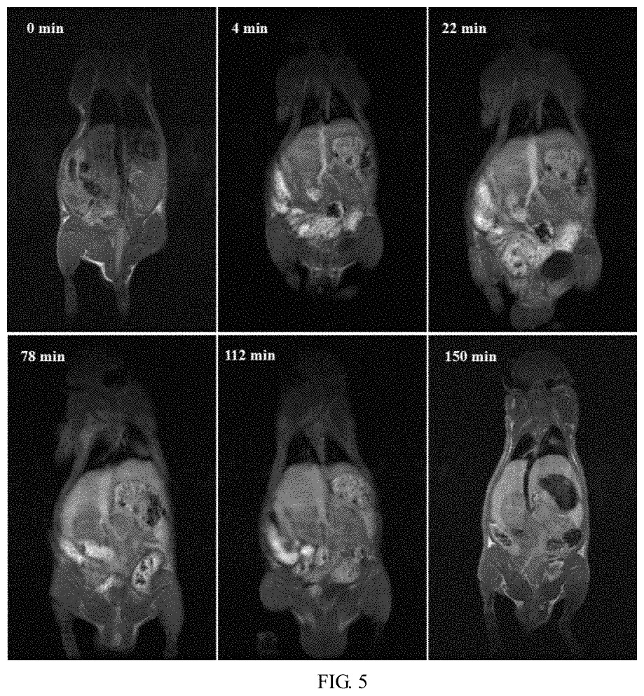

After the mice were anesthetized with isoflurane gas, the prepared micelle normal saline solution was injected through the tail vein of the mice. The magnetic resonance angiography of the micelle serving as the Fe³⁺ magnetic resonance contrast agent in the mice body is shown in FIG. 5. It can be seen from FIG. 5 that within 0-30 minutes after injection of the contrast agent, the signal intensity of the mice blood vessel rapidly rises and reaches a peak value, and the mice vascular structure can be clearly observed. Then, the signal intensity of the blood vessel gradually decreases, and the blood vessel is completely cleared when about 150 min after injection, which indicates that the circulation metabolism of the amphiphilic polymer nano micelle probe containing poly-3,4-dihydroxyphenylalanine (PDOPA) chelated ferric ions in the blood vessels is completed. Compared with the commercial Gd³⁺ reagent which cycles for less than 60 min in the body, the Fe³⁺ magnetic resonance contrast agent provided by the disclosure can make up the defect of short in-vivo circulation time.

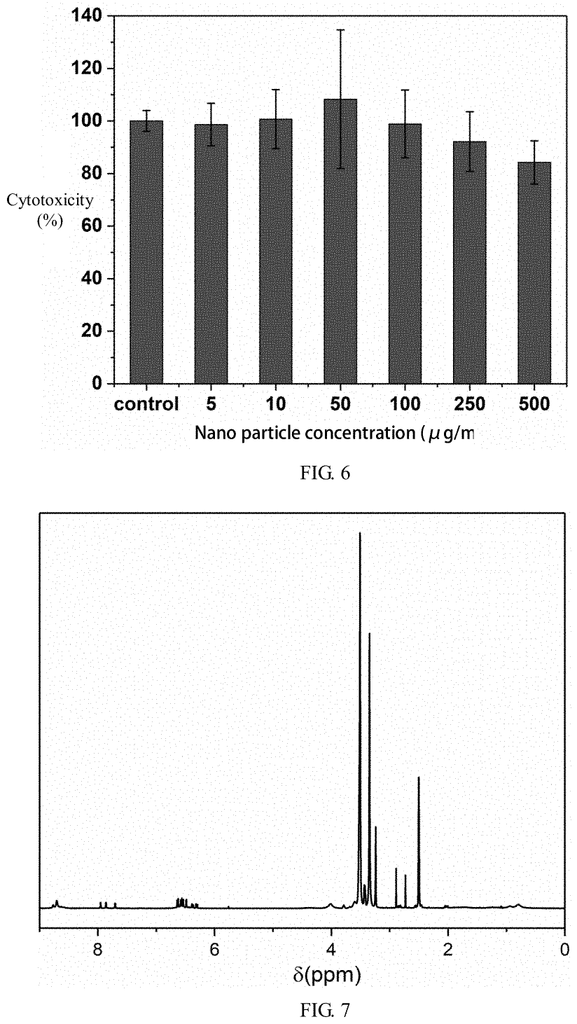

The cytotoxicity of the micelle is determined by MTT method, and 5 parallel samples are set for each sample. The cytotoxicity test results are shown in FIG. 6, which shows that all samples show very small cytotoxicity at the concentration of 5-500 μg/mL. When the concentration is greater than 50 μg/mL, the cell survival rate slightly decreases with the increase of concentration, but all are kept to be above 85%, indicating that this Fe³⁺ magnetic resonance contrast agent has low biotoxicity and good hiocompatihility.

### Example 2

(1) Other preparation conditions are the same as those in example 1. The difference is that amine-endcapped polyethylene glycol is used as a macromolecular initiator, and the structural formula of the prepared poly-3,4-dihydroxyphenylalanine-polyethylene glycol block copolymer (PDOPA-b-PEG) initiator is shown in the following formula:

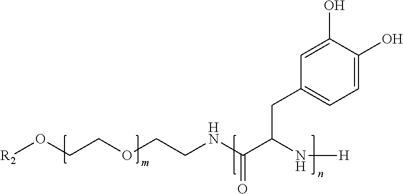

wherein, R₂ is methyl; m=5˜200, and n=5˜50.

(2) 9.7 mg of weighed P′DOPA-b-PEG was dissolved with DMF to be prepared into a solution, and then DMF solution containing 3.27 mg of Fe(NO₃)₃.9H₂O was added slowly, and the above mixture solution was dialyzed in deionized water for 48 hours. The obtained micelle solution was subjected to metered volume and used after being filtered with a filter film having a pore size of 0.45 μm.

Other performance test conditions are the same as those in example 1, and the micelle has an average particle size of 30 nm, and has an MRI in-vitro enhancement effect.

### Example 3

(1) The structural formula of poly-3,4-dihydroxyphenylalanine-polyoligoethyleneglycol methacrylate grafted polymer (POEGMA-g-PDOPA) is as follows:

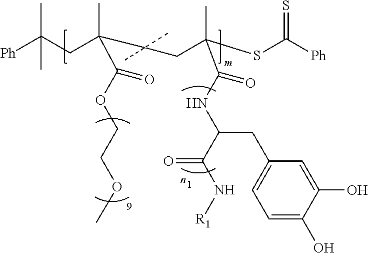

wherein, R₁ is n-butyl; m=5˜200, n₁=5˜50;

The specific synthesis steps include:

PDOPA was prepared by triggering ring opening polymerization and deprotection of CBZ-protected dopa NCA via n-butylamine, and the conditions are the same as those in example 1; polyoligoethyleneglycol methacrylate (POEGMA) was prepared through RAFT polymerization. 247.4 mg of POEGMA and 134.0 mg of PDOPA were dissolved in 1 mL of DMF, and reacted for 4 days in 35° C. oil bath. The polymer solution was poured into ether to be precipitated, filtered and dried in vacuum for 1 day, so as to obtain the poly-3,4-dihydroxyphenylatanine-polyoligoethyteneglycolmethacrylate grafted polymer. The proton NMR spectrum of the polymer is shown in FIG. 7.

(2) 22.7 mg of weighed POEGMA-g-PDOPA was dissolved with DMF to be prepared into a solution, and then the DMF solution containing 5.83 mg of Fe(NO₃)₃.9H₂O was added slowly, and the above mixture solution was dialyzed in deionized water for 48 hours. The obtained micelle solution was filtered with a filter film having a pore size of 0.45 pin and subjected to metered volume.

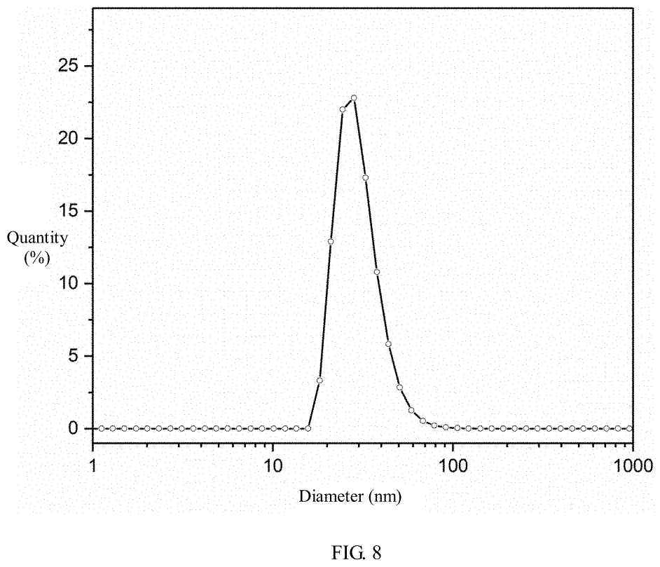

Other performance test conditions are the same as those in example 1. DLS test results are shown in FIG. 8. The micelle has an average particle size of 30 nm, and has an MRI in-vitro enhancement effect.

### Example 4

(1) Other preparation conditions are the same as those in example 1. The difference is that a polypropylenimine tetramine dendrimer (generation 1) is used as an initiator, and the structural formula of the prepared poly-3,4-dihydroxyphenylalanine-polysarcosine star polymer (PDOPA-b-PSar star copolymer) is as follows:

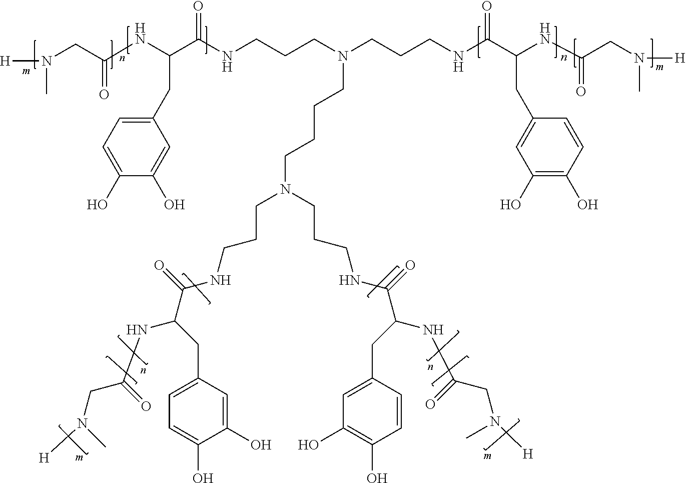

wherein, M=5˜200, and n=5˜50.

(2) 10.1 mg of weighed PDOPA-b-PSar star copolymer was dissolved with DMF to be prepared into a solution, and then DMF solution containing 2.21 mg of Fe(NO₃)₃.9H₂O was added slowly, and the obtained mixture solution was dialyzed in deionized water for 48 hours. The micelle solution was filtered with a filter film having a pore size of 0.45 μm and subjected to metered volume.

Other performance test conditions are the same as those in example 1, and the micelle has an average particle size of 35 nm, and has an MRI in-vitro enhancement effect.

We claim:

1. An amphiphilic polymer nano micelle containing poly-3,4-dihydroxyphenylalanine chelated ferric ions, wherein a hydrophobic block in the amphiphilic polymer nano micelle is a poly-3,4-dihydroxyphenylalanine block; a catechol functional group on a side chain of a biodegradable poly-3,4-dihydroxyphenylalanine block chelates ferric ions, and a chelating bond is as shown in the following formula:

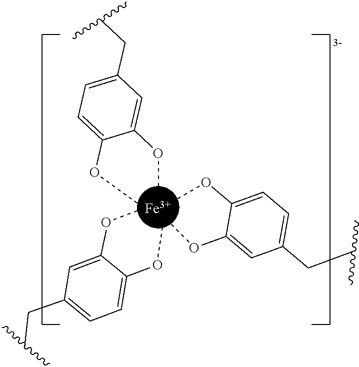

2. The amphiphilic polymer nano micelle containing poly-3,4-dihydroxyphenylalanine chelated ferric ions according to claim 1, wherein a hydrophilic block in the amphiphilic polymer is a polysarcosine block, a polyethylene glycol block, a polyoligoethyleneglycol methacrylate block, a polyvinyl alcohol block, or a polyacrylic acid block.

3. The amphiphilic polymer nano micelle containing poly-3,4-dihydroxyphenylalanine chelated ferric ions according to claim 1, wherein the chain length of the hydrophilic block in the amphiphilic polymer is 1˜1500; the chain length of poly-3,4-dihydroxyphenylalanine block is 1˜500.

4. The amphiphilic polymer nano micelle containing poly-3,4-dihydroxyphenylalanine chelated ferric ions according to claim 1, wherein the amphiphilic polymer is a diblock, triblock, multiblock, random, star-shaped, annular or grafted polymer containing the poly-3,4-dihydroxyphenylalanine block.

5. The amphiphilic polymer nano micelle containing poly-3,4-dihydroxyphenylalanine chelated ferric ions according to claim 1, wherein the amphiphilic polymer has any one of the following structure formulas (1)˜(5): 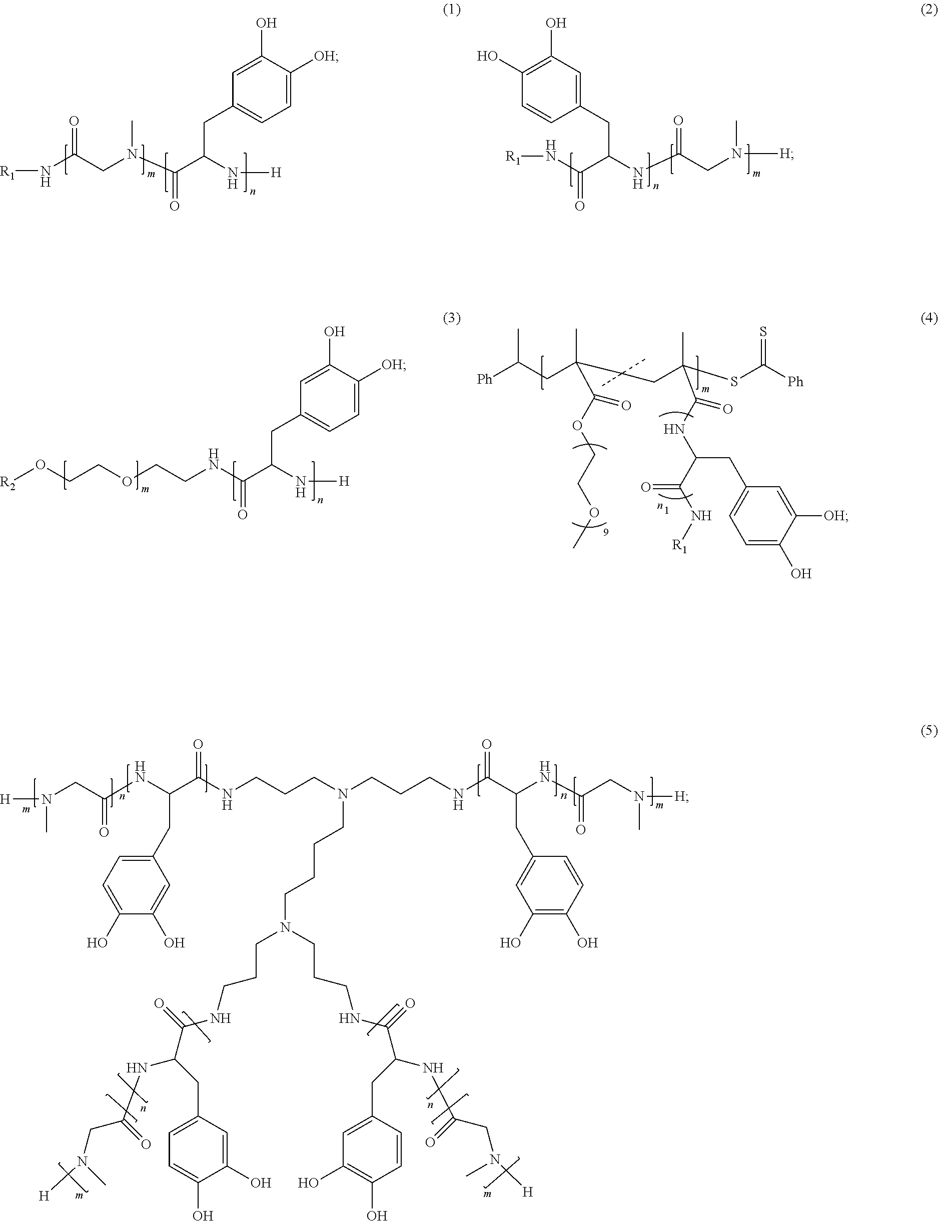
wherein, R₁ is independently selected from alkyl, benzyl and a silyl group; R₂ is independently selected from alkyl; m is an integer of 1˜1500, n is an integer of 1˜500, and n₁ is an integer of 1˜200.

6. The amphiphilic polymer nano micelle containing poly-3,4-dihydroxyphenylalanine chelated ferric ions according to claim 5, wherein the chain length of the poly-3,4-dihydroxyphenylalanine is between 5 and 50; the chain length of polysarcosine or polyethylene glycol is between 5 and 200.

7. Use of an amphiphilic polymer nano micelle containing poly-3,4-dihydroxyphenylalanine chelated ferric ions according to claim 1 as a magnetic resonance T₁ imaging contrast agent in the field of magnetic resonance imaging.

8. The use according to claim 7, wherein the magnetic resonance contrast agent comprises the amphiphilic polymer nano micelle containing poly-3,4-dihydroxyphenylalanine chelated ferric ions, a pharmaceutical excipient, a diluent or ingredients.

9. The use according to claim 7, wherein the average particle size of the magnetic resonance contrast agent is 20˜200 nm; the amount of the chelated ferric ions is 10˜1000 ppm.
